(19) United States
(12) Patent Application Publication
(10) Pub. No.: US 2010/0056958 A1
(43) Pub. Date: Mar. 4, 2010

(54) DEVICE FOR MEASURING BLOCKAGE LENGTH IN A BLOOD VESSEL

(76) Inventor: Kumar Lava Ravi, Paradise Valley, AZ (US)

Correspondence Address: PATENTMETRIX, 14252 CULVER DR. BOX 914, IRVINE, CA, 92604, US

(21) Appl. No.: 12/486,755

(22) Filed: Jun. 18, 2009

Related U.S. Application Data
(60) Provisional application No. 61/073,407, filed on Jun. 18, 2008.

(51) Int. Cl. A61B 5/107

(52) U.S. Cl. 600/587

(57) ABSTRACT

The present invention is directed toward a lesion measuring device for use with cardiac catheterization assemblies. In one embodiment, the assembly comprises a plurality of rulers physically attached to at least one housing. The housing is configured to connect the assembly to a catheterization assembly. The assembly includes a slidable member engaged with at least one of the plurality of rulers, and a torquer configured to move in relation to a guidewire and the slidable member.

### CROSS-REFERENCE TO RELATED APPLICATIONS

The present invention relies on U.S. Provisional Patent Application No. 61/073,407, of the same title, which was filed on Jun. 18, 2008.

### FIELD OF THE INVENTION

This invention generally relates to medical diagnostics, and particularly relates to an apparatus for measuring physical characteristics, such as length, of arteries, veins, and other lumens of the body. More particularly, this invention relates to a device for measuring length of a region of plaque or blockage in a blood vessel.

### BACKGROUND OF THE INVENTION

In the field of medical diagnostics and treatment, cardiologists often treat patients with conditions affecting blood flow in vessels near the heart. A commonly used procedure to restore blood flow through cardiac arteries narrowed by plaque deposits or other obstructions (coronary artery blockage), is balloon catheterization (angioplasty). In this particular procedure, an inflatable balloon is fed into the cardiac artery and inflated to dilate the affected length of artery. After dilation, a stent, which is a thin scaffold or support typically made of plastic or metal and formed in the shape of a perforated tube, is delivered and installed within the blood vessel to maintain an increased blood flow through the cross-sectional flow path. In certain configurations, the stent is medicated.

One of the difficulties cardiologists encounter in this procedure is the measurement of the length of the affected region of blood vessel. These measurements are critical, because they are used to determine the stent size appropriate for the length of blockage. If the stent size is not appropriate for a given blockage, the patient may require insertion of multiple stents. This is unwarranted as it would be highly invasive to the patient and would involve the risk of multiple surgeries. Thus, accurate sizing of the blockage is important to ensure the success of the procedure.

Several techniques have been employed in the prior art for measuring blockage. In one conventional technique, measurement is made using a balloon (angioplasty). A guide wire is first advanced through the guiding catheter, into the patient's coronary artery until it reaches the affected area. A dilatation catheter, having an inflatable balloon on the distal portion thereof (wherein the distal portion is the portion that is guided away from the practitioner and into the patient), is advanced into the patient's coronary artery over the previously introduced guide wire until the balloon of the dilatation catheter is properly positioned across the blockage. Typically, in order to see the positioning of the balloon, the balloon is equipped with fixed metal markers, that can be detected using X-ray technologies. Traditionally, these metal markers are compared to the length of blockage. Once properly positioned, the dilatation balloon is inflated to a predetermined size at relatively high pressures so that the stenosis is compressed against the arterial wall and the wall expanded to open up the vascular passageway.

One problem or disadvantage with this conventional technique, however, is determining reasonable distances between markers on the wire that would yield reasonable measurement accuracy. For example, with a 10 mm separation between markers, the resolution available to measure lesions is not good, while a 5 mm distance may prove to be too close. Further, if a guide wire with markers is used and then a balloon/stent with markers passed along with the guide wire, it becomes confusing for the observer to distinguish the several markers in the image. Thus, markers can serve as a distraction when looking at the lesion.

In another conventional approach, a cardiologist reviews X-ray images of the heart after contrast material is introduced into the bloodstream of the patient. In this case, the cardiologist must rely on experience and training to make a judgment regarding the size of the affected length. As such, this method may not always provide repeatability, accuracy, and precision in the measurement of the length. In yet another conventional method, ultrasonic transducers are fed into the patient's arteries via a catheter, for imaging the vessel walls to estimate the length. This device is very expensive and the necessary procedure is very time consuming.

Other methods are also employed for measuring blockage length in a body lumen. For example, U.S. Pat. No. 6,450,976, assigned to Accumed Systems, Inc., describes an “[a]pparatus for measuring length in a body lumen in conjunction with a catheterization assembly having a larger-diameter stationary component and a smaller-diameter moveable component with at least a distal end of the moveable component terminating in a radio-opaque marker, the apparatus comprising: an outer barrel including a large-diameter clamp adapted for retaining the larger-diameter stationary component of the catheterization assembly; an inner barrel, slideably disposed within the outer barrel, the inner barrel including a small-diameter clamp adapted for retaining the smaller-diameter moveable component of the catheterization assembly; and a scale operative to show a relative position of the inner barrel with respect to the outer barrel, thereby indicating the relative position of the moveable component within the body lumen.” This device, however, is quite complicated to deploy, as it is a two-component device used for measuring both length and diameter of lesion. In addition, there is no reasonable measure of accuracy for this device.

In addition, U.S. Pat. No. 6,428,512, assigned to Advanced Cardiovascular Systems Inc., describes “[a] guidewire for performing an intraluminal procedure, comprising: a. an elongated core having a proximal section with a proximal portion configured to extend out of the patient during an intraluminal procedure and a distally tapered distal section; b. a flexible body disposed about the distal section of the elongated core; c. a radiopaque marker on a distal part of the guidewire; and d. an arcuate member slidably mounted on the proximal portion of the proximal section of the core having a plurality of indicia disposed thereon, the position of said arcuate member on the proximal portion being adjustable to a first location relative to a reference point to represent a first end of an intracorporeal length to be measured when the distal marker is adjacent to the first end; and moving with the proximal portion of the core member when the guidewire is moved so that the distal marker is adjacent to a second end of the intracorporeal length to determine the distance from the indicia representing the first location for the distal marker to a second location on the arcuate member which represents a second location of the distal marker so as to thereby determine the distance between the two end of the intracorporeal length.” This device, however, employs “[a] wheeled distance sensing member” that “may be pressed into engagement with the surface of the proximal end of the guidewire extending out the patient. The wheel of the distance sensing member rotates as the guidewire is moved and this rotation can be converted into a suitable distance readout. Similarly, an electro-optical system may be utilized to measure the distance the guidewire moves.”

Conventional methods and apparatuses for measuring the length or size of blockage in blood vessels are thus cumbersome and/or expensive. Further, they do not provide sufficient precision of measurement. Additionally, most of these procedures are highly invasive and carry the risks associated with any other invasive procedure.

Therefore, what is needed is a simple, minimally invasive, and accurate method and apparatus for measurement of lesions in blood vessels.

What is also needed is a simple, minimally invasive, and accurate method and apparatus for measurement of the length of lesions in blood vessels.

### SUMMARY OF THE INVENTION

The present invention is directed toward a lesion measuring device for use with cardiac catheterization assemblies. In one embodiment, the assembly comprises a plurality of rulers physically attached to at least one housing, wherein said housing is configured to connect the assembly to a catheterization assembly, a slidable member engaged with at least one of said plurality of rulers, and a torquer configured to move in relation to a guidewire and said slidable member.

Optionally, the catheterization assembly is a Touhy-Borst Y-adapter. The housing comprises a band. The band is rubberized. The housing comprises a ring coupler. The ring is rubberized. The assembly comprises at least two rulers. The at least two rulers are substantially parallel. The at least two rulers are spaced to accommodate a guide wire between them. The slidable member comprises a slot for said guidewire.

In another embodiment, the assembly comprises a plurality of rulers physically attached to at least one housing, wherein said housing is configured to connect the assembly to a catheterization assembly, a member engaged with at least one of said plurality of rulers, and a torquer having a slot, wherein said slot is configured to receive to a guidewire and wherein said torquer is configured to move in relation to said member.

Optionally, the catheterization assembly is a Touhy-Borst Y-adapter. The housing comprises a band. The housing comprises a ring coupler. The assembly comprises at least two rulers. The at least two rulers are substantially parallel. The at least two rulers are spaced to accommodate a guide wire between them. The assembly further comprises a second member. The member and second member are slidable and configured to physically slide along a length of at least one of said plurality of rulers. The member is slidable, perpendicular to at least one of said plurality of rules, and configured to physically slide along a length of said at least one of said plurality of rulers.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 depicts one embodiment of the device of the present invention for measuring length of a lesion or blockage in a blood vessel;

FIG. 2 is a perspective view illustrating the measurement mechanism of the device of the present invention;

FIG. 3 illustrates another embodiment of the device of the present invention for measuring length of a lesion or blockage in a blood vessel; and

FIG. 4 is a flow chart illustrating operational steps of one embodiment of the present invention.

### DETAILED DESCRIPTION OF THE INVENTION

The present invention is directed toward a novel method for measuring length of a region of plaque or blockage in a blood vessel. The present invention employs a measuring device external to the patient (extracorporeal) for measuring the length of a vascular lesion that eliminates the use of radio-markers on the wire itself.

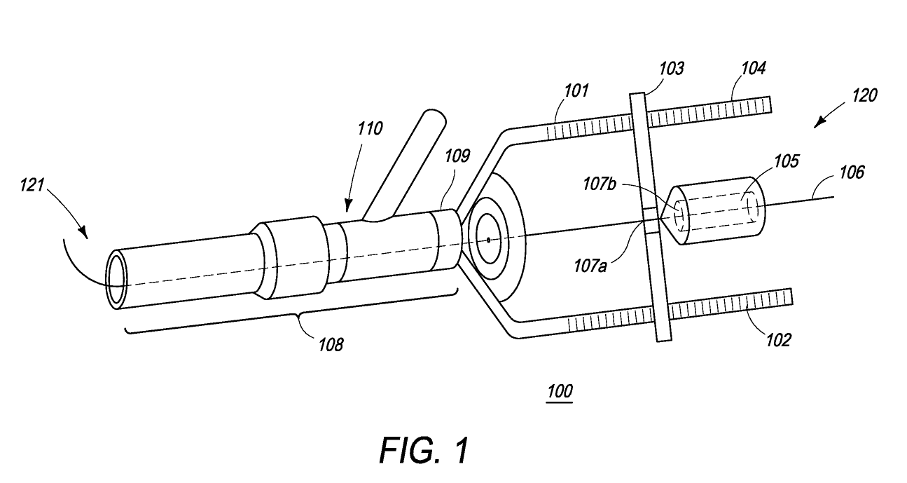

FIG. 1 depicts one embodiment of the device of the present invention for measuring length of a lesion or blockage in a blood vessel. In one embodiment, device 100 comprises a first fixed horizontal ruler 101 and a second fixed horizontal ruler 102 and vertical sliding sleeve 103. In one embodiment, first and second horizontal rulers 101 and 102, respectively, are marked in units of length. It should be understood to those of ordinary skill in the art that the ruler markings are scaled, or placed at fixed intervals, so that they can be used as a reference standard in measurement. For example, and not limited to such example, when the horizontal rulers are used to measure arterial blockage, it is preferred that the units of length marked are in millimeters, with additional markings for every tenth of a millimeter. These markings serve as a visual reference representing the displacement that occurs when the vertical sliding sleeve 103 slides over the horizontal rulers 101, 102 during the measurement of blockage.

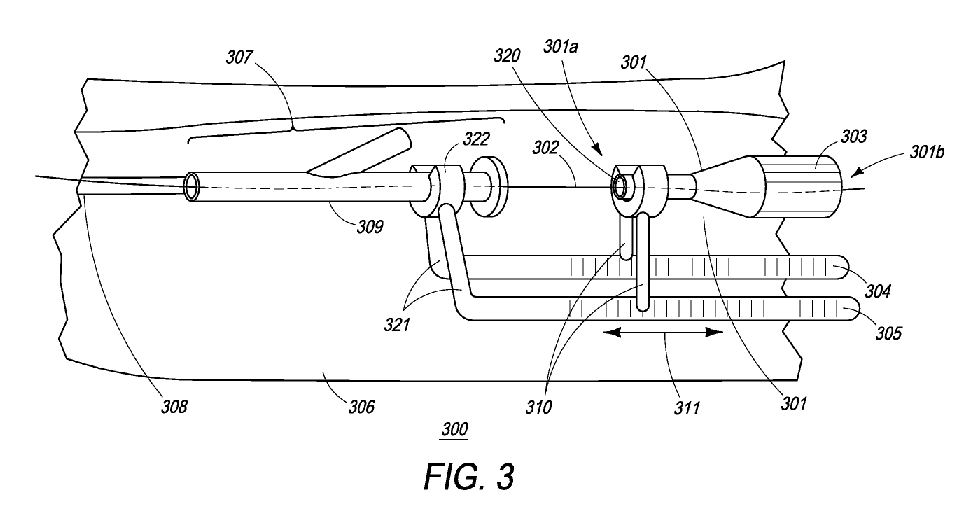

In one embodiment, first horizontal ruler 101 and second horizontal ruler 102 are parallel to each other, and thus, lie in the same plane, and are also co-planar with, and separated by, a guide wire 106, as shown in FIG. 1. In another embodiment, the horizontal rulers 101, 102 are parallel to each other but not separated by the guide wire 106, permitting the device to rest more comfortably on the patient's leg (thigh platform), usually 40-50 cm from the groin. This embodiment is illustrated, and described in further detail, with respect to FIG. 3.

The device 100 also comprises a torquer 105, at proximal end 120 (the end that is close to the clinician), which is used to 1) set a reference point A for the measurement and 2) optionally control the sliding movement of the vertical sleeve 103. The torquer 105 also allows for controlling (or limiting) the movement of guide wire 106. The vertical sleeve 103 is, in one embodiment, provided with a slot 107a to allow the guide wire 106 to pass through unobstructed and the torque is, in one embodiment, provided with a slot 107b to allow the guide wire to pass through. Using the torquer 105, guide wire 106 can be advanced out of the distal end 121 (the end that is close to the patient) of a catheter 108 into the patient's coronary artery to reach the affected area or blockage in the artery. One of ordinary skill in the art would appreciate that device 100 may be used with any conventional catheterization assembly 108.

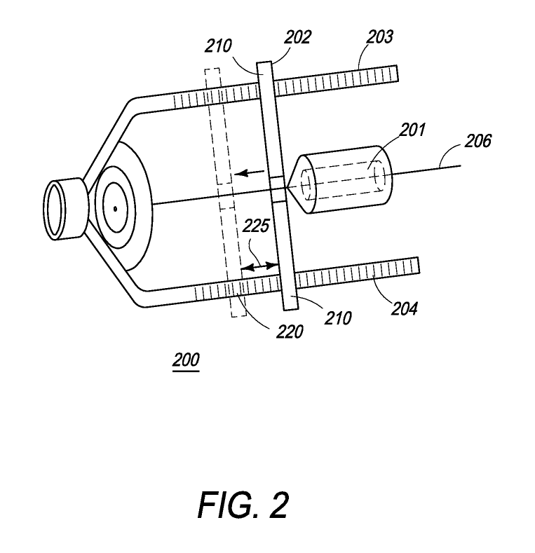

Measurement device 100 further comprises an adapter, a ring, band, rubberized band, rubberized ring, or band-like member 109 (shown in more detail in FIG. 2) for coupling the measurement device to a standard catheterization assembly 108. A suitable adapter 110 used in cardiac catheterization procedures, such as a Touhy-Borst Y-adapter with side arm and valve, couples with the ring-like member 109, to attach the device 100 to the catheter assembly 108.

The torquer 105 is a standard piece of equipment that can be used to manipulate the guide wire externally (from outside the patient). The torquer 105 comprises a screw mechanism (not shown), which when tightened, acts to fix the movement of guide wire 106. Thus, when the practitioner tightens the screw on the torque 105, fixed reference point A is created. It should be noted herein, and will be described in further detail later, that the torquer can be tightened at any point to create fixed reference point A, including, but not limited to a tip, transition between visible/invisible regions, markers, and the like.

FIG. 2 is a perspective view illustrating the measurement mechanism 200 of the device of the present invention. A practitioner is able to see x-ray images of the heart after contrast material is introduced into the bloodstream of the patient. In order to use the measurement device of the present invention, the practitioner passes a guide wire 206 through the blood vessel of concern, which can be viewed by X-ray imaging. As the guide wire 206 passes through, the practitioner can decide upon a reference point in the X-ray image of the blood vessel, to mark the starting point of the region of interest. The reference point can be any discernable part of the image such as the tip of guide wire, transition between a visible and an invisible part in the image, a marker on the wire, and the like.

Referring to FIG. 2, as soon as the guide wire 206 reaches this reference point, the practitioner tightens the torquer 201. This acts to acts to a) create reference point A, 210 and b) fix the movement of the guide wire (shown in FIG. 1). The point on the horizontal rulers 203 and 204 where the vertical sliding sleeve 202 is positioned upon tightening the torque is referred to as fixed reference point A 210. This point is marked and/or noted by the practitioner. It should be noted that the practitioner can mark the position of the vertical sliding sleeve 202 by any suitable marking means, including a clip, ink marker, label, or the like.

Thereafter, the torquer is operated such that the guide wire is advanced further through the lesion. Thus, as soon as the torquer is fixed to create a reference point A, and the position of the sliding sleeve 202 is marked, the movable portion of the device is pushed forward. In one embodiment, the movable portion comprises both the torquer and vertical sliding sleeve. In one embodiment, the torquer and vertical sliding sleeve are independently movable. The space between the two horizontal rulers 203 and 204 is large enough to allow for easy manipulation of the torquer 201. As the guide wire advances, the slidable vertical sleeve 202 is also pushed forward along with the guide wire over the two horizontal rulers 203 and 204. It should be noted that, in one embodiment, the torquer 201 is used to push vertical sliding sleeve forward. In another embodiment, the practitioner pushes the vertical sliding sleeve forward, as necessary, to mark points on the rulers. When the guide wire 206 reaches at the end of lesion or blockage, the practitioner again tightens the torquer 201, to fix the movement of guide wire (not shown). The position of the vertical ruler 202 on the horizontal rulers 203 and 204 determines point B 220. The practitioner marks the point B 220, and the distance 225 between point A 210 and point B on the horizontal rulers 203 and 204 provides a measure of the length of the lesion or blockage in the blood vessel. Thus in the present invention, the start point and end point for measurement are based on the torque point, that is the fixed point created using the torque mechanism.

One of ordinary skill in the art would appreciate that the mechanism for measuring length as illustrated in the present invention may not only be employed to measure a blockage in the coronary artery, but can be used to measure the length of any region of interest in a blood vessel through which a guide wire may be passed.

In one embodiment, the torquer is integrated with a housing in such a manner that it reduces the movement of the guide wire. This serves to improve the precision of measurement as any undesirable small movement or shake in movement of the wire is prevented. This embodiment is illustrated in FIG. 3. Referring to FIG. 3, the device 300 comprises a housing 301, having a distal end 301a and a proximal end 301b, where the distal end 301a is closest to the catheterization assembly 307 and the proximal end 301b comprises a torquer 303. Further, distal end 301a of housing 301 further comprises an opening 320 for guide wire 302 to be inserted through.

Device 300 further comprises at least one vertical sliding sleeve 310, and preferably two vertical sliding sleeves 310, attached to and thus, integrated with, housing 301 at its distal end 301a. The device 300 further comprises a first horizontal measurement ruler 304 and a second horizontal measurement ruler 305, which are parallel to each other, and which preferably rest on the patient's thigh 306. Vertical sliding sleeves 310 are perpendicular to, and slide over horizontal rulers 304 and 305. Vertical sliding sleeves 310 are used to mark reference points, and thus, provide a measurement of the distance 311 travelled by the guide wire 302 between the two marked reference points, and hence the length of the region of interest.

Horizontal rulers 304 and 305 are attached to, at their distal end 321, a ring or collar connector 322. As mentioned previously, the measurement device 300 of the present invention is coupled to a catheterization assembly 307, at the distal end 321 of the horizontal rulers by means of attaching a ring or collar connecter 322 to a suitable Y-adapter 309. The catheter 307 provides entry for the guide wire 302 into the patient at point 308.

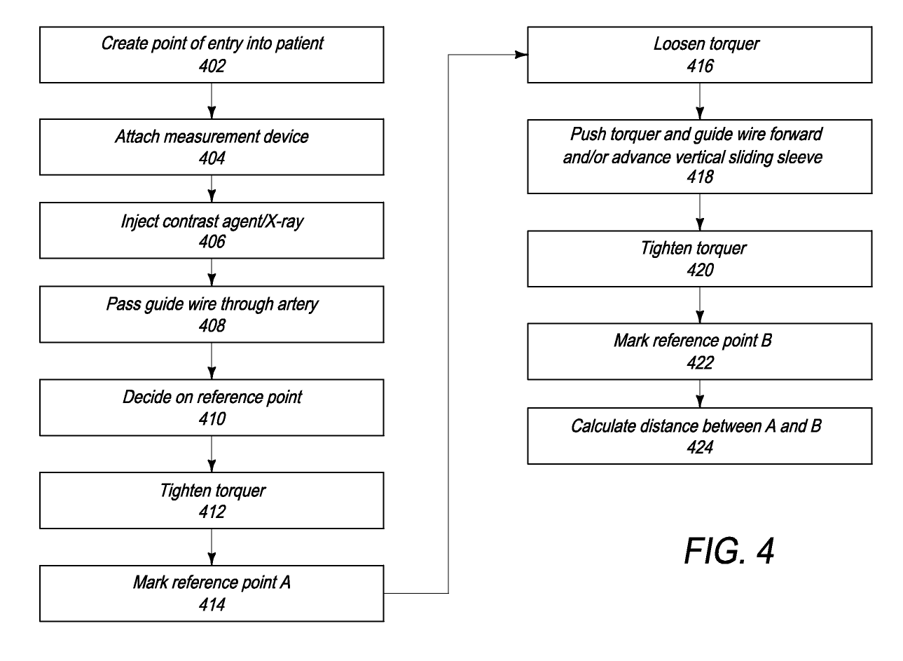

FIG. 4 is a flow chart illustrating operational steps of one embodiment of the present invention. Now referring to FIG. 4, in step 402, a point of entry is created in a patient via a standard catheter. In step 404, the measurement device of the present invention is coupled to the standard catheterization assembly using, as described above, a ring or band-like member. A suitable adapter used in cardiac catheterization procedures, such as a Touhy-Borst Y-adapter with side arm and valve, couples with the ring-like member to attach the device to the catheter assembly. In step 406, the practitioner injects the patient artery with a contrast material so that he can simultaneously view X-ray images of the heart. In order to use the measurement device of the present invention, in step 408, the practitioner passes a guide wire through the blood vessel of concern, which can be viewed by the X-ray imaging. As the guide wire passes through, the practitioner, in step 410, decides upon a reference point in the X-ray image of the blood vessel, to mark the starting point of the region of interest. The reference point can be any discernable part of the image such as the tip of guide wire, transition between a visible and an invisible part in the image, a marker on the wire, and the like.

As soon as the guide wire reaches this reference point, in step 412, the practitioner tightens the torque to a) create reference a point A and b) fix the movement of the guide wire (shown in FIG. 1). In one embodiment, the torque moves the vertical sliding sleeve into position and thus, the point on the horizontal rulers where the vertical sliding sleeve is positioned upon tightening the torque is referred to as fixed reference point A. In another embodiment, the practitioner may optionally move the vertical sliding sleeve to the appropriate position after tightening the torque. This is especially convenient in the case where the measurement is being made in reverse, that is, from the end of the region of interest to the beginning of the region of interest, or as the guidewire is being removed from the patient. Thus, the torquer is not required to move the vertical sliding sleeve. Fixed reference point A is marked and/or noted by the practitioner, in step 414. It should be noted that the practitioner can mark the position of the vertical sliding sleeve by any suitable marking means, including a clip, ink marker, label, or the like.

Thereafter, the torquer is operated such that the guide wire is advanced further through the lesion. Thus, as soon as the torquer is fixed to create reference point A, and the position of the vertical sliding sleeve is marked and/or recorded, the torquer is loosened in step 416, to allow the movable portion of the device to be pushed forward in step 418. As the guide wire is advanced using the torquer, the slidable vertical sleeve is also pushed forward along with the guide wire over the twin horizontal rulers. It should be noted that, in one embodiment, the torquer is used to push the vertical sliding sleeve forward. In another embodiment, the practitioner pushes the vertical sliding sleeve forward, as necessary, to mark points on the rulers. When the guide wire reaches the end of lesion or blockage, the practitioner again tightens the torquer in step 420, to fix the movement of guide wire (not shown). The position of the vertical ruler on the horizontal rulers determines point B. The practitioner marks point B in step 422. The practitioner then calculates, in step 424, the distance between point A and point B on the horizontal rulers to provide a measure of the length of the lesion or blockage in the blood vessel.

The device of the present invention can be used to measure the length of a region of interest in a blood vessel, in case when the guide wire is going in as well as when it is coming out. This is because the measurement relies on an external reference point, which is the fixed point created using the torquer as described above.

The device or product of the present invention is designed to be disposable in order to help maintain better hygiene in the catheterization procedures where it is to be used. Further, the device can be easily coupled to existing catheterization mechanisms, and is designed to conveniently snap in and out of the existing side arm and valve of the adapters commonly used in cardiac procedures. This lends additional operational simplicity to the device of the present invention.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

We claim:

1. An assembly comprising:
a. A plurality of rulers physically attached to at least one housing, wherein said housing is configured to connect the assembly to a catheterization assembly.
b. A slidable member engaged with at least one of said plurality of rulers; and
c. A torquer configured to move in relation to a guidewire and said slidable member.

2. The assembly of claim 1 wherein said catheterization assembly is a Touhy-Borst Y-adapter.

3. The assembly of claim 1 wherein the housing comprises a band.

4. The assembly of claim 3 wherein the band is rubberized.

5. The assembly of claim 1 wherein the housing comprises a ring coupler.

6. The assembly of claim 5 wherein the ring is rubberized.

7. The assembly of claim 1 comprising at least two rulers.

8. The assembly of claim 7 wherein the at least two rulers are substantially parallel.

9. The assembly of claim 8 wherein the at least two rulers are spaced to accommodate a guide wire between them.

10. The assembly of claim 1 wherein the slidable member comprises a slot for said guidewire.

11. An assembly comprising:
a. A plurality of rulers physically attached to at least one housing, wherein said housing is configured to connect the assembly to a catheterization assembly.
b. A member engaged with at least one of said plurality of rulers; and
c. A torquer having a slot, wherein said slot is configured to receive to a guidewire and wherein said torquer is configured to move in relation to said member.

12. The assembly of claim 11 wherein said catheterization assembly is a Touhy-Borst Y-adapter.

13. The assembly of claim 11 wherein the housing comprises a band.

14. The assembly of claim 11 wherein the housing comprises a ring coupler.

15. The assembly of claim 11 comprising at least two rulers.

16. The assembly of claim 15 wherein the at least two rulers are substantially parallel.

17. The assembly of claim 16 wherein the at least two rulers are spaced to accommodate a guide wire between them.

18. The assembly of claim 11 further comprising a second member.

19. The assembly of claim 18 wherein said member and second member are slidable and configured to physically slide along a length of at least one of said plurality of rulers.

20. The assembly of claim 11 wherein said member is slidable, perpendicular to at least one of said plurality of rules, and configured to physically slide along a length of said at least one of said plurality of rulers.
